(19) United States
(12) Patent Application Publication
(10) Pub. No.: US 2015/0010959 A1
(43) Pub. Date: Jan. 8, 2015

(54) METHOD FOR PRODUCING SACCHARIDES CONTAINING GLUCOSE AS MAIN COMPONENT

(71) Applicants: Daisuke Taneda, Ibaraki (JP); Makoto Ikeo, Ibaraki (JP); Shohei Okino, Ibaraki (JP)

(72) Inventors: Daisuke Taneda, Ibaraki (JP); Makoto Ikeo, Ibaraki (JP); Shohei Okino, Ibaraki (JP)

(73) Assignee: JGC CORPORATION, TOKYO (JP)

(21) Appl. No.: 14/375,436

(22) PCT Filed: Feb. 16, 2012
(86) PCT No.: PCT/JP2012/053636

§ 371 Date: Jul. 30, 2014

(51) Int. Cl. C12P 19/14; C12P 19/02

(52) U.S. Cl. CPC C12P 19/14; C12P 19/02
USPC 435/99

(57) ABSTRACT

A method for producing saccharides containing glucose as a main component is described, including degrading cellulose and/or hemicellulose with a cellulase, wherein an additive containing a protein and an amino acid and/or a yeast lysate solution is added to the cellulose and/or hemicellulose and the cellulase is used to cause an enzymatic saccharification reaction of saccharifying the cellulose and/or hemicellulose.

### TECHNICAL FIELD

This invention relates to a method for producing saccharides containing glucose as a main component, the method using an enzymatic saccharification reaction of enzymatically degrading cellulose and/or hemicellulose contained in biomass to generate saccharides containing glucose as a main component.

### BACKGROUND ART

Currently, techniques for producing bioethanol from cellulose-based biomass serving as a raw material are being studied in various countries around the world.

Studies on methods for producing bioethanol from cellulose-based biomass serving as a raw material are directed toward various methods. The most promising technique being studied toward practical implementation all over the world is an enzymatic ethanol production technique of enzymatically hydrolyzing cellulose and/or hemicellulose. The major issue for putting the enzymatic ethanol production technique into practical use is reducing the amount of the enzyme used. In order to address this issue, high-performance enzymes and pretreatment techniques for biomass have been developed.

In the development of high-performance enzymes, methods were devised in which microorganisms (such as fungi) that produce enzymes are modified by gene recombination technology to achieve production of high-performance enzymes.

In the development of pretreatment techniques for biomass, methods were devised in which lignin that is one of components constituting biomass is broken down or dissolved with dilute sulfuric acid, ammonia, high-temperature water, or the like, so that the contact efficiency between cellulose and enzymes is increased, the crystalline structure of cellulose is changed to an amorphous structure, and the rate and yield of degrading cellulose by enzymes are increased.

However, even these methods do not allow a sufficient reduction in the amount of the enzyme used. Thus, the technique still incurs high cost and has not reached the stage of practical use.

When cellulose and/or hemicellulose contained in biomass is degraded with an enzyme to produce saccharides containing glucose as a main component, a part of the enzyme added is adsorbed on lignin that is one of components constituting biomass. It is known that such an enzyme adsorbed on lignin is not likely to become detached from lignin and hence does not play a role in hydrolyzing cellulose and/or hemicellulose to produce saccharides. Such a phenomenon is called unproductive enzyme adsorption on lignin. If this unproductive adsorption can be suppressed, the amount of the enzyme used can be reduced.

A method for suppressing the unproductive adsorption under study is a method in which, during degradation of cellulose and/or hemicellulose in biomass with an enzyme, an additive that suppresses unproductive adsorption of the enzyme is added to the reaction system of the biomass and the enzyme.

Known examples of the additive include surfactants, polyethylene glycol, bovine serum albumin (refer to Non-Patent Literature 1, for example), and skim milk (refer to Patent Literature 1, for example).

### CITATION LIST

### Patent Literature

Patent Literature 1: Japanese Unexamined Patent Application Publication No. 2009-72144

### Non-Patent Literature

Non-Patent Literature 1: Kumar R, Wyman C E. Effect of additives on the digestibility of corn stover following pretreatment by leading technologies. Biotechnol Bioeng 2009; 102: 1544-1557

### SUMMARY OF INVENTION

### Technical Problem

However, such known additives including surfactants, polyethylene glycol, bovine serum albumin, and skim milk exert the effect on some types of biomass, but, in some other cases, exert no or little effect.

In addition, these additives are expensive and hence use of the additives does not contribute to considerable reduction in the cost of the enzymatic ethanol production technique.

This invention has been made in view of the above-described circumstances. An object of this invention is to provide a method for producing saccharides containing glucose as a main component, in which the amount of an enzyme used is reduced by using an additive that is inexpensive, easily available, and considerably effective.

### Solution to Problem

A method for producing saccharides containing glucose as a main component according to this invention includes degrading cellulose and/or hemicellulose with cellulase, wherein an additive containing a protein and an amino acid and/or a yeast lysate solution is added to the cellulose and/or hemicellulose, and the cellulase is used to cause an enzymatic saccharification reaction of saccharifying the cellulose and/or hemicellulose.

In the method for producing saccharides containing glucose as a main component according to this invention, the protein and the amino acid are preferably derived from grain or constitute whey.

In the method for producing saccharides containing glucose as a main component according to this invention, the protein and the amino acid derived from grain are preferably generated from grain as waste or a secondary product in a starch producing plant, an ethanol producing plant, a flour milling plant, a vegetable oil producing plant, or a brewery.

In the method for producing saccharides containing glucose as a main component according to this invention, the yeast lysate solution is preferably a solution containing a protein and an amino acid derived from yeast useful in ethanol fermentation of saccharides, the solution being obtained by lysing, with alkali, the yeast.

In the method for producing saccharides containing glucose as a main component according to this invention, a metal and a surfactant are preferably added to the cellulose and/or hemicellulose.

In the method for producing saccharides containing glucose as a main component according to this invention, the metal is preferably a metal mixture containing at least two metals selected from the group consisting of iron, zinc, manganese, and copper.

### Advantageous Effects of Invention

In a method for producing saccharides containing glucose as a main component according to this invention, an additive containing a protein and an amino acid and/or a yeast lysate solution is added to the reaction system of cellulose-based biomass raw material and cellulase. Thus, such a protein and an amino acid contained in the additive are adsorbed on lignin, so that unproductive adsorption of cellulase on lignin can be suppressed during saccharification of the cellulose raw material with cellulase. As a result, the amount of cellulase that plays a role in hydrolyzing the cellulose and/or hemicellulose in the cellulose-based biomass raw material can be increased so that the reaction rate can be increased and the amount of the enzyme used can be reduced. In addition, the protein and the amino acid derived from grain, an alkali solution in which yeast is lysed with alkali, and whey are inexpensive, easily available, and exert a strong effect on addition, compared with known additives in the existing literatures, patents, and the like.

### BRIEF DESCRIPTION OF DRAWINGS

FIG. 1 is a graph illustrating the relationships between the reaction time of enzymatic degradation and the concentration of glucose generated in Experimental examples 1 to 4.

FIG. 2 is a graph illustrating the relationships between the reaction time of enzymatic degradation and the concentration of glucose generated in Experimental examples 7 to 11.

### DESCRIPTION OF EMBODIMENTS

Methods for producing saccharides containing glucose as a main component according to embodiments of this invention will be described.

These embodiments are specifically described for facilitating understanding of the gist of the invention and do not limit this invention unless otherwise specified.

In a method for producing saccharides containing glucose as a main component according to an embodiment, an additive containing a protein and an amino acid and/or a yeast lysate solution is added to the reaction system of cellulose and/or hemicellulose and cellulase, and the cellulase is used to cause an enzymatic saccharification reaction of saccharifying the cellulose and/or hemicellulose.

Hereinafter, cellulose and hemicellulose and cellulose-containing biomass are sometimes collectively referred to as cellulose raw material.

Examples of the cellulose raw material include 1) raw materials obtained by subjecting biomass (wood, grass, and agricultural residues) to a treatment of breaking down or dissolving lignin contained in the biomass and to a pretreatment of partially breaking the crystalline structure of cellulose (turned into an amorphous structure); 2) waste raw materials containing cellulose as a main component, such as waste paper, corrugated cardboard, and papermaking sludge; and 3) cotton fiber waste such as shirts and towels.

The above-described pretreatment step may be performed by subjecting biomass to, for example, an alkali treatment, an organic solvent treatment, a dilute sulfuric acid treatment, a hot water treatment, or an explosion treatment.

In some cases, such a pretreatment step may be unnecessary for waste raw materials such as waste paper, corrugated cardboard, and papermaking sludge; and cotton fiber waste such as shirts and towels.

In the method for producing saccharides containing glucose as a main component according to this embodiment, an additive containing a protein and an amino acid and/or a yeast lysate solution is added to the cellulose raw material, and then an enzyme that hydrolyzes cellulose and/or hemicellulose (cellulase) is added. Alternatively, the additive and cellulase are mixed in advance and the resultant solution mixture is added to the cellulose raw material.

The protein and the amino acid are derived from grain or constitute whey.

The protein and the amino acid derived from grain are generated from grain such as corn, wheat, potato, or rice as waste or a secondary product in, for example, a starch producing plant, an ethanol producing plant, or a brewery.

The grain-derived protein and amino acid are added to the cellulose raw material in the following manner: an aqueous solution that is generated in a starch producing plant or the like and contains grain-derived protein and amino acid is adjusted so as to have an appropriate concentration, and the resultant solution is added. Alternatively, an aqueous solution containing grain-derived protein and amino acid is dried so as to be turned into powder, this powder is dissolved in water to prepare an aqueous solution containing the grain-derived protein and amino acid, and this solution is added to the cellulose raw material. Thus, the grain-derived protein and amino acid can be adsorbed uniformly over the entirety of the cellulose raw material.

The solution in which yeast is lysed with alkali is inexpensively and easily obtained by lysing yeast generated as waste from ethanol fermentation of saccharides.

Such alkali lysis is performed under conditions including: use of an aqueous solution of sodium hydroxide, a temperature of 50° C. to 100° C., and a pH of 9 to 14 during lysis. After alkali lysis, an acid such as sulfuric acid is added to adjust the pH of the solution to be in the range of 4 to 6, and this solution is used as the additive.

Whey, which is also referred to as milk serum, is an aqueous solution obtained as a result of removal of milk fat, casein, and the like from milk. Whey is waste generated during production of processed food such as cheese from milk serving as a raw material. When whey is used as the additive, whey is adjusted so as to have an appropriate concentration and then added. Alternatively, whey is dried so as to be turned into powder, and this powder is dissolved in water and used as the additive.

The above-described additives may be added alone or in combination of two or more thereof to the cellulose raw material. The type of the additive added to the cellulose raw material is appropriately selected according to, for example, the type of cellulose raw material.

In addition to such an additive, a metal and a surfactant are preferably added to the cellulose raw material.

The metal added to cellulose raw material may be a metal mixture containing at least two metals selected from the group consisting of iron, zinc, manganese, and copper. Of these metals, in view of price, a metal mixture containing iron and zinc is preferred.

Examples of the surfactant added to cellulose raw material include Tween 80 (trade name, produced by Tokyo Chemical Industry Co., Ltd.), Tween 20 (trade name, produced by Tokyo Chemical Industry Co., Ltd.), and polyethylene glycol. Among these surfactants, in view of performance regarding the efficiency of reduction in the amount of the enzyme used, Tween 80 is preferred.

Subsequently, a reaction vessel (enzymatic degradation vessel) is charged with the cellulose raw material, an aqueous solution (enzyme aqueous solution) containing an appropriate amount of a cellulase for degradation of the cellulose raw material, and the above-described additive; and the cellulose raw material, the enzyme aqueous solution, and the additive are mixed (preparation step). The additive is added before or at the same time when the enzyme aqueous solution is added to the cellulose raw material. Alternatively, a solution prepared by mixing the additive and the enzyme aqueous solution in advance is added.

In this preparation step, the pH of the reaction vessel solution is adjusted so as to satisfy a pH condition optimal for the enzyme used. In addition, the temperature of the reaction vessel is adjusted so as to satisfy a temperature condition optimal for the enzyme used.

In this preparation step, the pH of the mixture of the cellulose raw material, the enzyme aqueous solution, and the additive is preferably adjusted such that the enzyme actively functions. Specifically, the pH of the aqueous solution in the reaction system is preferably adjusted to be in the range of 4 to 6.

In this preparation step, the temperature of the mixture is preferably adjusted such that the enzyme actively functions. Specifically, the temperature of the reaction system is preferably increased to be in the range of 40° C. to 60° C.

The concentration of the cellulose raw material in the reaction vessel is preferably 5 g to 50 g in 100 mL of the solution, that is, 5 w/v % to 50 w/v %, and more preferably 10 g to 30 g in 100 mL of the solution, that is, 10 w/v % to 30 w/v %.

The cellulase is used as an enzyme for degrading cellulose raw material.

When the cellulose raw material has a large content of hemicellulose, in addition to the cellulase, an enzyme that degrades hemicellulose such as a xylanase or a mannanase is preferably added.

The mixture is stirred with, for example, a stirring blade.

In this embodiment, the mixture in the reaction vessel is slowly stirred and mixed such that the enzyme in the enzyme aqueous solution is not excessively deactivated, so that the enzymatic saccharification of the cellulose raw material (cellulose and/or hemicellulose) is efficiently achieved.

In this enzymatic saccharification reaction step, the temperature of the mixture is preferably adjusted such that the enzyme actively functions. Specifically, the temperature is preferably maintained at 40° C. to 60° C.

The enzymatic saccharification reaction step is performed until the enzymatic saccharification of the cellulose raw material has sufficiently proceeded and the reaction no longer proceeds. For example, the enzymatic degradation of the cellulose raw material is performed at 40° C. to 60° C. for about 2 to about 20 days.

In a method for producing saccharides containing glucose as a main component according to the embodiment, an additive containing a protein and an amino acid and/or a yeast lysate solution is added to the cellulose raw material. Thus, the additive is adsorbed on lignin in the cellulose raw material, so that enzyme adsorption on lignin can be suppressed during the saccharification reaction of the cellulose raw material with the enzyme. As a result, the amount of the enzyme that plays a role in hydrolyzing the cellulose raw material can be increased, so that the reaction rate can be increased and the amount of the enzyme used can be reduced.

Hereinafter, this invention is described in further details with Experimental Examples. However, this invention is not limited to the following Experimental Examples.

### EXPERIMENTAL EXAMPLE 1

Papermaking sludge was degraded with a cellulase under the following experiment conditions.
Weight of the papermaking sludge: 10 g
Amount of the cellulase added: 60 mg—protein
Amount of the solution: 100 mL
Temperature: 50° C.
pH: 5

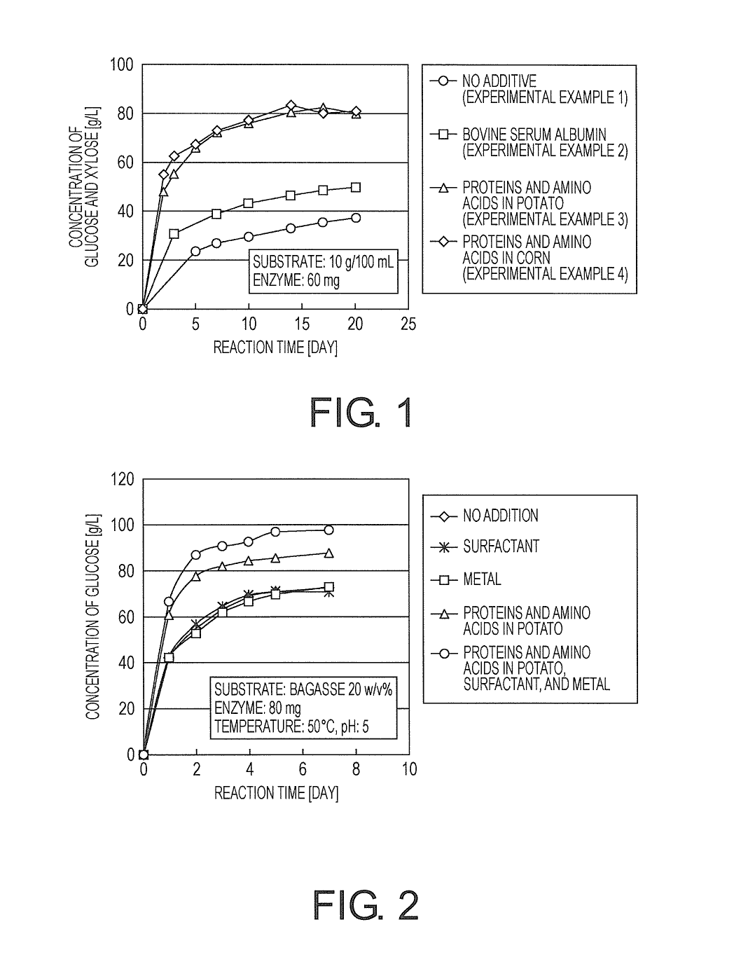

The relationship between the reaction time of the enzymatic degradation and the total monosaccharide concentration in terms of glucose and xylose generated was determined. The result is illustrated in FIG. 1.

### EXPERIMENTAL EXAMPLE 2

Papermaking sludge was mixed with bovine serum albumin serving as an additive and was degraded with a cellulase under the following experiment conditions.
Weight of the papermaking sludge: 10 g
Amount of the cellulase added: 60 mg—protein
Amount of the bovine serum albumin added: 20 mg—protein/g—papermaking sludge
Amount of solution: 100 mL
Temperature: 50° C.
pH: 5

The relationship between the reaction time of the enzymatic degradation and the total monosaccharide concentration in terms of glucose and xylose generated was determined. The result is illustrated in FIG. 1.

### EXPERIMENTAL EXAMPLE 3

Papermaking sludge was mixed with proteins and amino acids in potato serving as an additive and was degraded with a cellulase under the following experiment conditions.
Weight of the papermaking sludge: 10 g
Amount of the cellulase added: 60 mg—protein
Amount of the potato protein added: 20 mg—protein/g—papermaking sludge
Amount of solution: 100 mL
Temperature: 50° C.
pH: 5

The relationship between the reaction time of the enzymatic degradation and the total monosaccharide concentration in terms of glucose and xylose generated was determined. The result is illustrated in FIG. 1.

The proteins and amino acids of potato were those remaining in a solution obtained by mashing potato and separating the starch and residue thereof.

### EXPERIMENTAL EXAMPLE 4

Papermaking sludge was mixed with proteins and amino acids in corn serving as an additive and was degraded with a cellulase under the following experiment conditions.
Weight of the papermaking sludge: 10 g
Amount of the cellulase added: 60 mg—protein
Amount of the corn protein added: 20 mg—protein/g—papermaking sludge
Amount of the solution: 100 mL
Temperature: 50° C.
pH: 5

The relationship between the reaction time of the enzymatic degradation and the total monosaccharide concentration in terms of glucose and xylose generated was determined. The result is illustrated in FIG. 1.

The proteins and amino acids of corn were those remaining in a solution obtained by mashing corn and separating the starch and residue thereof.

According to the results in FIG. 1, as the total monosaccharide concentration in terms of glucose and xylose in Experimental Example 1 in which no additive was added is compared with the total monosaccharide concentration in terms of glucose and xylose in Experimental Example 2 in which bovine serum albumin was added as an additive, the saccharide concentration was slightly higher in the result of Experimental Example 2 than in the result of Experimental Example 1. Thus, the effect provided by adding bovine serum albumin is weak. In contrast, in Experimental Example 3 in which the proteins and amino acids of potato were added and in Experimental Example 4 in which the proteins and amino acids of corn were added, such additions resulted in considerably high total monosaccharide concentrations in terms of glucose and xylose generated. These results indicated that addition of proteins and amino acids derived from potato or corn allows considerable enhancement of the enzymatic saccharification performance.

### EXPERIMENTAL EXAMPLES 5-1 to 5-3

Papermaking sludge was mixed with a surfactant, a solution obtained by lysing yeast with alkali, or whey each serving as an additive and was degraded with a cellulase under the following experiment conditions.
Weight of the papermaking sludge: 10 g
Amount of the cellulase added: 60 mg—protein
Surfactant (trade name: Tween 80, produced by Tokyo Chemical Industry Co., Ltd.): 0.02 wt % (ratio of the amount added to the amount of the solution)
Amount of the alkali-lysis yeast solution added: 20 mg—protein/g—papermaking sludge
Amount of the whey added: 20 mg—protein/g—papermaking sludge
Amount of the solution: 100 mL
Temperature: 50° C.
pH: 5

Table 1 shows the total monosaccharide concentrations in terms of glucose and xylose on the 14^{th} day from the initiation of the enzymatic reaction. The results in Table 1 indicate that the effect provided by adding the surfactant is weak. In contrast, in the case where the alkali-lysis yeast solution or whey was added, the total monosaccharide concentrations in terms of glucose and xylose are increased, which indicates promotion of the enzymatic saccharification reaction.

TABLE 1

| | Experimental | Experimental | Experimental | Experimental |
|---|---|---|---|---|
| | Example 1 | Example 5-1 | Example 5-2 | Example 5-3 |
| Additive | None | Surfactant | Alkali-lysis | Whey |
| | | | yeast | |
| | | | solution | |
| Concentration | 33.5 | 38.2 | 76.5 | 80.3 |
| of generated | | | | |
| monosaccha- | | | | |
| rides (g/L) | | | | |

The results in FIG. 1 and Table 1 indicate the followings. Even in the case where bovine serum albumin or a surfactant that was verified by the past studies to provide the effect of suppressing unproductive enzyme adsorption was added, the effect provided by the addition was weak. However, by adding proteins and amino acids derived from grain such as potato or corn, an alkali-lysis yeast solution, or whey, the enzymatic saccharification reaction was considerably promoted.

This is probably because bovine serum albumin is a pure protein, whereas the three additives (grain-derived proteins, alkali-lysis yeast solution, and whey) added this time contain, in addition to proteins, amino acids and low-molecular-weight polymers of amino acids that provide synergistic effects in combination with the proteins.

### EXPERIMENTAL EXAMPLES 6-1 to 6-4

Eucalyptus having been subjected to an explosion pretreatment was mixed with proteins and amino acids contained in potato, an alkali-lysis yeast solution, or whey each serving as an additive and was degraded with a cellulase under the following experiment conditions.
Weight of the exploded eucalyptus: 20 g
Amount of the cellulase added: 40 mg—protein
Amount of the potato protein added: 20 mg—protein/g—exploded eucalyptus
Amount of the alkali-lysis yeast solution added: 20 mg—protein/g—exploded eucalyptus
Amount of the whey added: 20 mg—protein/g—exploded eucalyptus
Amount of the solution: 100 mL
Temperature: 50° C.
pH: 5

Table 2 shows glucose concentrations on the 14^{th} day from the initiation of the enzymatic reaction. The results in Table 2 indicate that, in a case where the proteins and amino acids in potato, the alkali-lysis yeast solution, or the whey was added, the glucose concentration was increased, which indicated promotion of the enzymatic saccharification reaction.

TABLE 2

| | Experimental | Experimental | Experimental | Experimental |
|---|---|---|---|---|
| | Example 6-1 | Example 6-2 | Example 6-3 | Example 6-4 |
| Additive | None | Potato | Alkali-lysis | Whey |
| | | protein | yeast | |
| | | | solution | |
| Concentration | 62.8 | 83.7 | 75.3 | 81.4 |
| of generated | | | | |
| monosaccha- | | | | |
| ride (g/L) | | | | |

### EXPERIMENTAL EXAMPLE 7

Bagasse was degraded with a cellulase under the following experiment conditions.
Weight of the bagasse: 20 g
Amount of the cellulase added: 80 mg—protein
Amount of the solution: 100 mL
Temperature: 50° C.
pH: 5

The relationship between the reaction time of the enzymatic degradation and the concentration of glucose generated was determined. The result is illustrated in FIG. 2.

### EXPERIMENTAL EXAMPLE 8

Bagasse was mixed with a surfactant serving as an additive and was degraded with a cellulase under the following experiment conditions.
Weight of the bagasse: 20 g
Amount of the cellulase added: 80 mg—protein
Surfactant (Tween-80): 0.1 wt % (ratio of the amount added to the amount of the solution)
Amount of the solution: 100 mL
Temperature: 50° C.
pH: 5

The relationship between the reaction time of the enzymatic degradation and the concentration of glucose generated was determined. The result is illustrated in FIG. 2.

### EXPERIMENTAL EXAMPLE 9

Bagasse was mixed with iron, copper, manganese, and zinc serving as an additive and was degraded with a cellulase under the following experiment conditions.
Weight of the bagasse: 20 g
Amount of the cellulase added: 80 mg—protein
20 μg of iron, 10 μg of copper, 2 μg of manganese, and 400 μg of zinc
Amount of the solution: 100 mL
Temperature: 50° C.
pH: 5

The relationship between the reaction time of the enzymatic degradation and the concentration of glucose generated was determined. The result is illustrated in FIG. 2.

### EXPERIMENTAL EXAMPLE 10

Bagasse was mixed with proteins and amino acids in potato serving as an additive and was degraded with a cellulase under the following experiment conditions.
Weight of the bagasse: 20 g
Amount of the cellulase added: 80 mg—protein
Amount of the potato protein added: 10 mg—protein/g—bagasse
Amount of the solution: 100 mL
Temperature: 50° C.
pH: 5

The relationship between the reaction time of the enzymatic degradation and the concentration of glucose generated was determined. The result is illustrated in FIG. 2.

The proteins and amino acids of potato were those remaining in a solution obtained by mashing potato and separating the starch and residue thereof.

### EXPERIMENTAL EXAMPLE 11

Bagasse was mixed with proteins and amino acids contained in potato, a surfactant, and iron serving as an additive and was degraded with a cellulase under the following experiment conditions.
Weight of the bagasse: 20 g
Amount of the cellulase added: 80 mg—protein
Amount of the potato protein added: 10 mg—protein/g—bagasse
Surfactant (trade name: Tween 80, produced by Tokyo Chemical Industry Co., Ltd.): 0.02 wt % by weight (ratio of the amount added to the amount of the solution)
20 μg of iron, 10 μg of copper, 2 μg of manganese, and 400 μg of zinc
Amount of the solution: 100 mL
Temperature: 50° C.
pH: 5

The relationship between the reaction time of the enzymatic degradation and the concentration of glucose generated was determined. The result is illustrated in FIG. 2.

The proteins and amino acids of potato were those remaining in a solution obtained by mashing potato and separating the starch and residue thereof.

As indicated by the results shown in FIG. 2, compared with Experimental Example 1 in which no additive was added, the glucose concentrations were not very different in Experimental Example 8 in which a surfactant was added as an additive and Experimental Example 9 in which metals were added as an additive. In contrast, the glucose concentration was considerably high in Experimental Example 11 in which proteins and amino acids contained in potato, a surfactant, and metals were added as an additive. In addition, this glucose concentration was higher than that in Experimental example 10 in which proteins and amino acids in potato were added as an additive. These results indicate that addition of proteins and amino acids contained in potato, a surfactant, and metals allows considerable enhancement of the enzymatic saccharification performance.

### INDUSTRIAL APPLICABILITY

This invention relates to a method for producing saccharides containing glucose as a main component by degrading cellulose and/or hemicellulose with a cellulase, wherein an additive containing a protein and an amino acid and/or a yeast lysate solution is added to cellulose and/or hemicellulose and the cellulase is used to cause an enzymatic saccharification reaction of saccharifying the cellulose and/or hemicellulose.

This invention allows production of saccharides containing glucose as a main component in which the amount of an enzyme used is reduced by using an additive that is inexpensive, easily available, and considerably effective.

1. A method for producing saccharides containing glucose as a main component by degrading cellulose and/or hemicellulose with a cellulase,
wherein an additive containing a protein and an amino acid and/or a yeast lysate solution is added to cellulose and/or hemicellulose and the cellulase is used to cause an enzymatic saccharification reaction of saccharifying the cellulose and/or hemicellulose.

2. The method according to claim 1, wherein the protein and the amino acid are derived from grain or constitute whey.

3. The method according to claim 2, wherein the protein and the amino acid derived from grain are generated from grain as waste or a secondary product in a starch producing plant, an ethanol producing plant, a flour milling plant, a vegetable oil producing plant, or a brewery.

4. The method according to claim 1, wherein the yeast lysate solution is a solution containing a protein and an amino acid derived from yeast useful in ethanol fermentation of saccharides, the solution being obtained by lysing, with alkali, the yeast.

5. The method according to claim 1, wherein a metal and a surfactant are added to the cellulose and/or hemicellulose.

6. The method according to claim 5, wherein the metal is a metal mixture containing at least two metals selected from the group consisting of iron, zinc, manganese, and copper.
